(19) United States
(12) Patent Application Publication
(10) Pub. No.: US 2023/0134003 A1
(43) Pub. Date: May 4, 2023

(54) METHOD AND APPARATUS FOR CLINICAL TESTING

(71) Applicant: Lightcast Discovery Ltd, Cambridge (GB)

(72) Inventors: Cameron FRAYLING, Cambridge (GB); Thomas Henry ISAAC, Cambridge (GB); Andreas Michael WAEBER, Cambridge (GB)

(73) Assignee: Lightcast Discovery Ltd, Cambridge (GB)

(21) Appl. No.: 17/767,730

(22) PCT Filed: Oct. 9, 2020
(86) PCT No.: PCT/GB2020/052508

§ 371 Date: Apr. 8, 2022

(30) Foreign Application Priority Data
Oct. 11, 2019 (GB) 1914724.8

(51) Int. Cl. C12Q 1/20; B01L 3/00

(52) U.S. Cl. CPC C12Q 1/20; B01L 3/50273; B01L 2200/0673; B01L 2300/0654; B01L 2400/0427

(57) ABSTRACT

A method for determining an interaction between a medicament and a cell type comprising an array of first microdroplets, each containing a cell type derived from a biological sample, an array of second microdroplets, each containing one or more medicaments at one or more predetermined concentrations, merging the array of first microdroplets and the array of second microdroplets to form an array of merged microdroplets, and monitoring the characteristics of one or more cells in the merged microdroplets using an optical detection system configured to detect an interaction between a cell type and a medicament.

### FIELD OF THE INVENTION

This invention relates to methods and apparatus for determining an interaction between a medicament and a cell of a cell type, and in particular to the rapid and accurate profiling of antimicrobial sensitivities and identities of infection-causing organisms.

### BACKGROUND TO THE INVENTION

The rapid and accurate profiling of infection-causing organisms is a significant challenge in healthcare. Blood culture analysis remains the gold standard for diagnosing infections such as sepsis. This method, however, is often too slow and cumbersome to affect any significant influence over the initial management of patients. Patients will often be treated immediately with a broad-spectrum antibiotic whilst the results of blood cultures are awaited. There is a need for a diagnostic test that is able to capture clinically relevant organisms and characterise antimicrobial resistance properties, such as the minimum inhibitory concentration (MIC) for a particular medicament, quickly.

Microfluidic platforms have been proposed for solving this need, however as yet no methods have been proven to be practical and reliable in a clinical setting. In particular, the ability to carry out simultaneous multiplexed assays on a plurality of different medicaments and cells has proven elusive.

Determination of MIC using microfluidics has been shown utilising continuous flow microfluidics, for example, by Choi et al, “Rapid antibiotic susceptibility testing by tracking single cell growth in a microfluidic agarose channel system”, Lab Chip, 2013, 13, 280-287. However, continuous flow microfluidics does not allow for the broad and elaborate panel of tests possible with microdroplet manipulation platforms.

Lyu et al, “Phenotyping antibiotic resistance with single-cell resolution for the detection of heteroresistance”, Sensors and Actuators B: Chemical 270 2018 369-404, describes a microfluidic method for the quantification of phenotypic heteroresistance by encapsulating single bacterial cells from a heterogeneous population into microfluidic droplets. However, the number of droplets that can be manipulated simultaneously, as well as the number of antibiotics and concentrations that can be evaluated simultaneously, are limited. The throughput is limited in part due to there being no mechanism for discarding empty droplets, which must instead be progressed through the method alongside those which contain cells.

Similar limitations are present in Sabhachandani et al, “Integrated microfluidic platform for rapid antimicrobial susceptibility testing and bacterial growth analysis using bead-based biosensor via fluorescence imaging”, Microchimica Acta 184 2017, 12, 4619-4628, which describes a droplet-based microfluidic method for phenotypic-based antimicrobial susceptibility testing (AST). Only a low viable droplet count could be achieved with no ability to remove unwanted droplets from the surface of the microfluidic chip.

There is a clear need for a method of characterizing antimicrobial resistance properties, such as MIC, that is quick and accurate enough to be practical in a clinical setting. Based on the current state of the art, no approaches have been proven to be able to do this reliably. It is within this context that the methods and apparatus of the present invention are set out.

### SUMMARY OF INVENTION

According to a first aspect of the present invention there is provided a method for determining an interaction between a medicament and a cell type, the method comprising:
providing an array of first microdroplets, each containing a cell type derived from a biological sample;
providing an array of second microdroplets, each containing one or more medicaments at one or more predetermined concentrations,
merging the array of first microdroplets and the array of second microdroplets to form an array of merged microdroplets;
monitoring the characteristics of one or more cells in the merged microdroplets using an optical detection system to detect an interaction between a cell type and a medicament.

In some embodiments the array of second microdroplets comprises microdroplets having a plurality of different concentrations for each medicament.

In some embodiments, each microdroplet may contain one or more cells.

In some embodiments, one or more cells can be of a single type or strain and/or a mixture.

In some embodiments the step of providing the array of first microdroplets comprises:
emulsifying the biological sample with an immiscible carrier fluid to form aqueous first microdroplets, at least some of which contain one or more cells of the cell type;
loading the first microdroplets onto a microfluidic chip configured to manipulate the microdroplets using real or virtual electrowetting electrodes;
determining that the cell-containing microdroplets contain at least one viable cell for proliferation; and
creating the array of first microdroplets from the at least one viable cell.

In some embodiments providing the array of first microdroplets comprises:
moving a first portion of the first microdroplets to a cell proliferation location configured to provide aerobic conditions;
moving a second portion of the first microdroplets to a second cell proliferation location, separate from the first cell proliferation location and configured to provide anaerobic conditions; and
monitoring and recording cell behaviour for the first and second portions of first microdroplets to determine whether the first microdroplets contain viable aerobic cells or viable anaerobic cells.

In some embodiments the environmental conditions at the first and second cell proliferation locations are selected to promote proliferation of cells of the cell type, the environmental conditions comprising temperature and ambient fluid composition.

In some embodiments the step of providing the array of second aqueous microdroplets comprises one or more of:
emulsifying one or more medicaments with an immiscible carrier fluid to form aqueous second microdroplets, each microdroplet containing at least one medicament type;
loading the second microdroplets onto a microfluidic chip configured to manipulate the microdroplets using real or virtual electrowetting electrodes;
performing a series of dilution operations on the second microdroplets to create an array.

In some embodiments the method further comprises performing an assay on a sub-sample of one or more cells from one or more microdroplets.

In some embodiments the assay is one or more of: a Gram stain, a destructive lysis assay, an oxidase test or a wheat germ agglutinin (WGA) assay.

In some embodiments the assay is performed prior to merging the first and second arrays.

In some embodiments the method further comprises performing at least one cell recognition assay on the cells from the biological sample utilising a classification algorithm.

In some embodiments the cell recognition step comprises analyzing one or more features of the cells from the biological sample, the one or more features comprising at least one of: cell motility, cell shape, and cell proliferation behaviour.

In some embodiments the method further comprises: based on characteristics of one or more cells detected in the merged microdroplets overtime, determining a minimum inhibitory concentration (MIC) of at least one medicament for cells of the cell type.

In some embodiments the medicament is an antibiotic or an antifungal medicament for treating sepsis.

In some embodiments the method further comprises determining an antibiotic or antifungal treatment regime for treating sepsis based at least in part on the determined MIC.

In some embodiments, the first and/or second microdroplets further comprise a growth media. The media can be a cell growth media and is selected from one or more of the following, but is not limited to, RPMI 1640, EMEM, DMEM, Ham’s F12, Ham’s F10, F12-K, HAT Medium, or modified versions thereof.

In some embodiments, the method may further comprise the step of splitting the merged microdroplets containing one or more cells to form a clonal colony.

In some embodiments, one or more cells can be stained for viability or detection studies and/or expresses a fluorescent protein. For example, a nucleic acid stain such as propidium iodide, TO-PRO™-3 Iodide, Zombie Green TM, or a membrane stain can be utilised. In some embodiments, nucleic acid or membrane stain can be used for counting number of cells. In some embodiments, death stain can be deployed for viability metrics.

According to another aspect of the present invention there is provided a microfluidic chip device configured to carry out the method of any preceding embodiment, the device comprising:
a sorting component configured to separate cell-containing microdroplets from empty microdroplets;
a microdroplet manipulation component configured to manipulate microdroplets using real or virtual electrowetting electrodes;
an optical detection system configured to monitor microdroplets contained in the microfluidic chip via one or more detection windows.

In some embodiments the device further comprises a cell-culturing component configured to hold the first microdroplets and provide conditions favourable for cell proliferation.

In some embodiments the device further comprises a sample preparation component configured to create a microdroplet emulsion in an immiscible carrier fluid from the biological sample.

In some embodiments, the detection system may be provided to detect one or more microdroplets. One or more microdroplets may utilize light or optical spectroscopy such as fluorescence spectroscopy. In some embodiments, a detector can be configured to detect the fluorescence of one or more microdroplets. In some embodiments, the detector can be a fluorescence detector.

In some embodiments the microdroplet manipulation component includes one or more OEWOD structures comprised of:
a first composite wall comprised of:
   a first substrate
   a first transparent conductor layer on the substrate, the first transparent conductor layer having a thickness in the range 70 to 250 nm; and
   a first dielectric layer on the photoactive layer, the first dielectric layer having a thickness in the range 30 to 280 nm;
a second composite wall comprised of:
   a second substrate;
   a second conductor layer on the substrate, the second conductor layer having a thickness in the range 70 to 250 nm;
   a photoactive layer activated by electromagnetic radiation in the wavelength range 400-850 nm on the second conductor layer, the photoactive layer having a thickness in the range 300-1500 nm and
   optionally a second dielectric layer on the second conductor layer, the second dielectric layer having a thickness in the range 30 to 280 nm
wherein the exposed surfaces of the first and second dielectric layers are disposed 1-180 µm apart to define a microfluidic space adapted to contain microdroplets;
an A/C source to provide a voltage across the first and second composite walls connecting the first and second conductor layers;
at least one source of electromagnetic radiation having an energy higher than the bandgap of the photoactive layer adapted to impinge on the photoactive layer to induce corresponding virtual electrowetting locations on the surface of the first dielectric layer; and
means for manipulating the points of impingement of the electromagnetic radiation on the photoactive layer so as to vary the disposition of the virtual electrowetting locations thereby creating at least one electrowetting pathway along which the microdroplets may be caused to move.

According to another aspect of the present invention, an array of microdroplets is provided, the array comprising:
a plurality of medicament microdroplets, each containing one or more medicaments from a panel of different medicament types at a range of predetermined concentrations; and
a number of control microdroplets which is less than or equal to the number of microdroplets in the plurality of medicament microdroplets, the control microdroplets containing no medicaments.

### BRIEF DESCRIPTION OF THE FIGURES

FIG. 1 shows a first part of an example experimental workflow, wherein sample cells are emulsified into a plurality of cell-containing microdroplets which are then loaded onto a microfluidic chip;

FIG. 2 shows a second part of the experimental workflow, wherein cell behaviour is monitored to identify viable cells suitable for the creation of an array of first microdroplets, and where an optional assay is carried out on one or more cells from the sample;

FIG. 3 shows a third part of the experimental workflow, wherein cultured cells are split into the array of first microdroplets;

FIG. 4 shows a fourth part of the experimental workflow, wherein a second array of microdroplets containing different concentrations of medicaments is prepared;

FIG. 5 shows a fifth part of the experimental workflow, wherein the first and second arrays are merged and cell behavior in the merged microdroplets is monitored to characterize the interaction;

FIG. 6 shows a side view of an example configuration of a microfluidic chip for carrying out the method of the present invention;

FIG. 7 shows a top-down view of an example configuration of the microfluidic chip of FIG. 6, and illustrates an example workflow which may be carried out thereon;

FIG. 8 shows an example array comprising a range of droplet sizes with a range of bacteria per droplet, produced according to the methods of the current invention.

FIG. 9 shows the experimental results of bacterial growth in microdroplets obtained according to the methods of the current invention.

FIG. 10 shows an example of a panel merge performed using methods and apparatus according to the present invention.

FIG. 11 shows a set of example experimental results obtained using methods and apparatus according to the present invention, which allowed for an MIC determination to be made between a strain of E. coli bacteria and gentamicin antibiotic.

### DETAILED DESCRIPTION OF THE INVENTION

In order to further explain various aspects of the present disclosure, specific embodiments of the present disclosure will now be described in detail in conjunction with the accompanying drawings.

The present invention provides methods and apparatus for determining an interaction between a cell and a medicament, and deriving information therefrom which may be used to develop a treatment regime using said medicament.

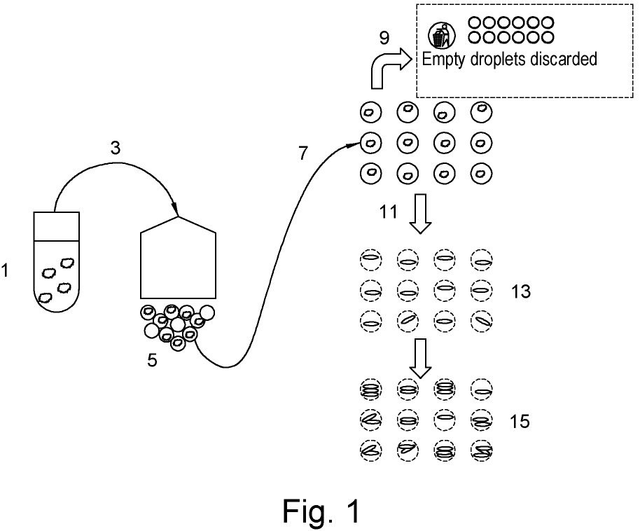

Referring to FIG. 1, a first part of an example workflow according to the method of the present invention is illustrated, the workflow comprising a number of steps. Although the steps of the example workflow are provided in a specific order, a person skilled in the art would understand that the method of the present invention could be carried out in any number of different orders.

In a first step 1, one or more sample cells are suspended in a nutrient solution, such as Luria-Bertani (LB) broth, tryptic soy broth (TSB), Mueller-Hinton broth or Super Optimal Broth (SOB).

In a second step 3 the cells are emulsified and dispensed into a fluorocarbon oil to form a plurality of microdroplets. Typically, the fluorocarbon oil is one of HFE-7500, HFE-7700, FC-40, or FC-70. Such oils are chosen to contain a suitable fluorinated surfactant such as RAN-008, Picosurf 1, Picosurf 2, or dSurf. The oil may optionally additionally be treated with nutrient solution to provide a more favourable environment to the emulsified cells. The person skilled in the art will understand that the specific nutrient solution used will depend upon the specific cell types under investigation.

Initially, the emulsion comprises both cell-containing microdroplets and empty microdroplets.

In a third step 5 the microdroplets are loaded onto a device such as a microfluidic chip. For example, the microdroplets may be loaded onto a chip comprising an optically-mediated electrowetting-on-device (oEWOD) stack structure.

In one example, the emulsifier is integrated into a device such as a microfluidic chip. For example, the emulsifier may be integrated into a chip comprising an optically-mediated electrowetting-on-device (oEWOD) stack structure.

In one example the emulsification is a vortex emulsification. In another example the emulsification is a step emulsification.

The microfluidic chip sorts 7 the first microdroplets into cell-containing microdroplets and empty microdroplets, with the empty microdroplets being discarded 9. Discarding the empty microdroplets may comprise removing them from the chip entirely or storing them in a holding area of the microfluidic chip. Sorting may be performed by optical inspection of each microdroplet. Each cell-containing microdroplet may contain one or more cells. In some examples, the sorting may be facilitated by automated software and microdroplets are manipulated along the surface of the microfluidic chip via oEWOD induced forces.

The cell-containing microdroplets are moved to a cell proliferation location 11 on the surface of the chip. The cell proliferation location may comprise a first cell proliferation location and a second cell proliferation location, the first cell proliferation location comprising a target region of the chip configured to provide environmental conditions favourable for aerobic cells to proliferate, and the second cell proliferation location comprising a target region of the chip configured to provide environmental conditions favourable for anaerobic cells to proliferate.

Once in position, the one or more cells are held in place at the target region 13. The cells are allowed to proliferate in a culturing step 15. In some embodiments, the culturing step requires the cells to be held in position at the one or more target regions for a particular length of time, which will depend on the generation time of the specific pathogen(s) under investigation. Under ideal experimental conditions, the culturing step may require the cells to be held in position between 15 minutes and 1 hour. It is recognized however that various factors may lengthen this incubation time in practice, however it is not expected that the culturing phase exceeds 12 hours. The person skilled in the art will understand that the specific length of time the cells are held at one or more target regions will vary depending on the exact type of cell under investigation and the number of generation cycles needed to acquire a sufficient number of sample cells.

At any point in the workflow, the cells contained in the microdroplets may be manipulated according to the needs of particular sampling assays in any number of ways. Such manipulation may comprise altering the electrowetting conditions for the microdroplets such that the microdroplets de-wet or partially de-wet from the surface. The term “de-wet” as used herein refers to the change in contact angle between the droplet and the chip surface such that the droplet is pulled away from the surface.

The oEWOD forces may also be used to agitate and “stir” the droplets to disperse the cells contained within, or to stretch and elongate the droplets to break off smaller, daughter droplets if it is desired to assay a single cell from a cultured colony. This process may be aided somewhat by the mother droplet remaining wetted or partially wetted to the surface of the target region. The daughter droplets may then be inspected for cell occupancy and, if the desired cell distribution is not achieved, the droplets may be re-merged and split once more.

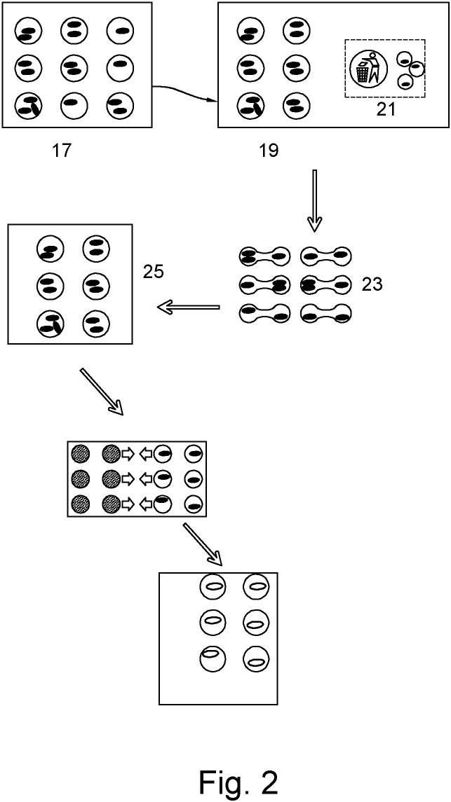

Referring to FIG. 2, after the cells have been allowed to proliferate for a sufficient length of time, the microdroplets are interrogated optically to determine which cells are viable for creating an array 17. In some embodiments, viable cells are those which are determined to have a suitable proliferation rate, and cells that are determined to have undergone few divisions are classified as non-viable. In other embodiments, cells that are determined to have undergone few divisions may be determined to be fastidious - that is, cells that are slow growing, or that will only grow if special nutrients are present in the growth medium or specific conditions met. In embodiments wherein fastidious cells are determined to be present, those cells may be incubated further under suitable conditions.

Microdroplets determined to contain viable cells are separated from the others 19 with the remaining non-viable cell-containing microdroplets being discarded 21, or incubated further as described above. If no cells have been determined to be viable at this point, the process is halted and either steps 1-17 are repeated using a new set of one or more sample cells, or the determination is made that the original sample contained no viable cells. In some embodiments where it is determined that the original sample contains no viable cells, the sample inspection is stopped and the result is reported as negative.

Optionally, if viable cells are determined to be present, the microdroplets containing those viable cells may be split 23 into two portions, with a first portion being subjected to an assay 25 for characterizing the cells contained therein, such as a Gram stain test or image-based cell identification, and the second portion being cultured further to generate a larger number of cells for more involved assays on-chip. The image-based cell identification may be carried out on the first or second portion of microdroplets or it may be carried out on all microdroplets containing viable cells without the need for the microdroplets to be split into two portions as in 23. This assay 25 may provide information useful for determining a set of medicaments to test against the cells.

In embodiments wherein an assay 25 is image-based cell identification, the image-based identification may be performed by a human or it may be performed automatically using a software algorithm. The person skilled in the art will understand that, using a database of known cells, it is possible to train a software algorithm to classify cells into known categories based on characteristics that the algorithm has derived from said database.

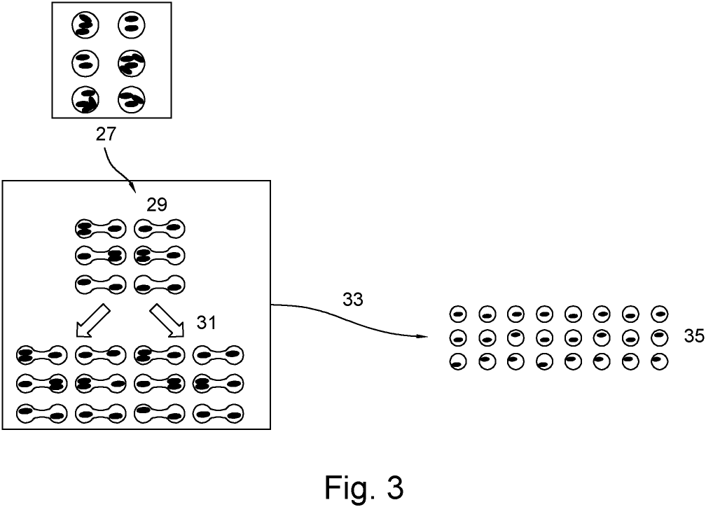

Referring to FIG. 3, the microdroplets containing viable cells are further cultured 27 in order to generate a sufficient number of cells to test a panel of medicaments of different types at varying concentrations. Once a sufficient number of cells are determined to have been generated, the microdroplets are subjected to a multi-way split 29 to obtain a plurality of first microdroplets each containing at least one viable cell, which are then formed into an array 31. Optionally, at this stage one or more microdroplets may be selected for further evaluation in the form of an assay, which may be in addition to or instead of 25. The microdroplets may be further split to build panels of daughter microdroplets each containing at least one cell. One or more microdroplets from an assay array comprising microdroplets containing appropriate assay reagents for the assay in question, at appropriate concentrations, may then be merged with the daughter microdroplets whilst the remaining microdroplets of 31 continue to be cultured.

The manipulations required to form the assay array may be performed on-chip or off-chip. In embodiments wherein the assay array is prepared off-chip, the assay array is then brought onto to the same chip as the first array. In embodiments wherein the assay array of microdroplets are prepared off-chip, the assay array may optionally be stored in a cartridge which is shared between multiple chips and which may dispense the required assay array on demand. In some embodiments the assay array may comprise a panel of pre-dropletised assay reagents stored on-chip, each assay reagent being accompanied by one or more control droplets containing no assay reagents.

In some embodiments the assay array of microdroplets is ‘pre-loaded’ onto the chip, and the chip is stored under conditions suitable for maintaining the integrity and activity of the assay reagents. Such conditions may, for example, be controlled temperatures. In some examples the temperature may be controlled to be approximately 4° C., selectively in the assay reagent storage region.

In some embodiments, the first microdroplets are further divided 33 such that the microdroplets in the array 35 each initially contain only a single viable cell.

In some embodiments, microdroplets containing viable cells may be split and merged with empty media-containing droplets iteratively whenever a minimum number of cells has been reached to prevent the cells from entering the stationary phase of the cell growth curve. The person skilled in the art will understand that the minimum number of cells at which microdroplets containing viable cells may be split and merged with empty-media containing droplets may vary depending on the specific cell type under investigation.

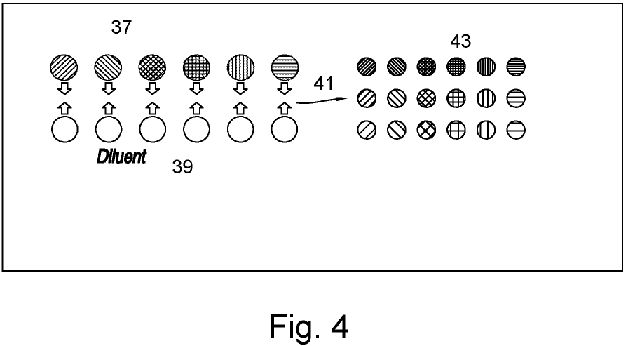

Referring to FIG. 4, separately to the array of first microdroplets, a second array of microdroplets 43 is formed by merging 41 microdroplets containing known concentrations/volumes of medicaments 37 with microdroplets containing known concentrations/volumes of diluent 39. Alternatively, the second array of microdroplets 43 may be formed by a series of alternating merge and split operations. Thus an array comprising microdroplets with known concentrations of medicaments, including microdroplets containing 0% concentration of medicaments to be used as controls, is created. As mentioned above, if an assay was carried out on the first microdroplets, the medicaments contained in the array of second microdroplets may be based on one or more results obtained from said assay.

The manipulations required to form the second array of microdroplets 43 may be performed on-chip or off-chip. In embodiments wherein the second array of microdroplets are prepared off-chip, the second array is then brought onto to the same chip as the first array. In embodiments wherein the second array of microdroplets are prepared off-chip, the second array may optionally be stored in a cartridge which is shared between multiple chips and which may dispense the required second array on demand. In some embodiments the second array may comprise a panel of pre-dropletised medicaments stored on-chip, each medicament being present at two or more dilution levels, and being accompanied by one or more control droplets containing no medicaments.

In some embodiments the second array of microdroplets is ‘pre-loaded’ onto the chip, and the chip is stored under conditions suitable for maintaining the integrity and pharmacological activity of the medicaments. Such conditions may, for example, be controlled temperatures. In some examples the temperature may be controlled to be approximately 4° C., selectively in the medicament storage region.

In some embodiments the second array comprises between one and ten different medicaments. In other embodiments, the second array comprises between one and fifteen different medicaments. In other embodiments, the second array comprises between one and twenty different medicaments.

In some embodiments the second array may comprise, for example, microdroplets containing different dilutions of one or more of oxacillin, vancomycin, imipenem, gentamicin, ciprofloxacin, cefoxitin, metronidazole, ampicillin, daptomycin, linezolid, nystatin or voriconazole.

The person skilled in the art will further understand that the second array may comprise one or more control microdroplets containing no medicament. These control microdroplets may further comprise the growth medium required for culturing of cells.

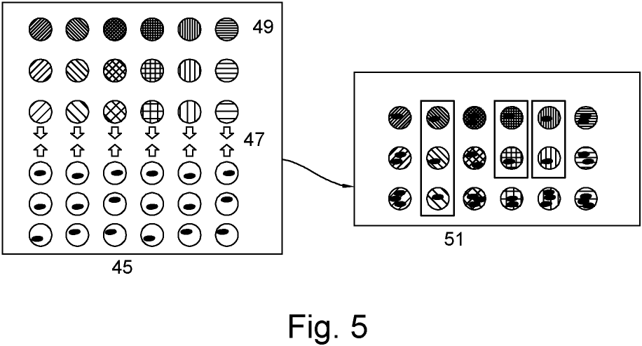

Referring to FIG. 5, the first array of microdroplets 45 is merged 47 with the second array of microdroplets 49. The merged array 51 is then cultured and the one or more cells contained within the merged microdroplets are monitored, optically or otherwise, to determine antimicrobial resistance properties of the cells. For example, the MIC for the cells in relation to each of the medicaments may be determined, and the medicaments may be determined to be bacteriostatic, bactericidal, fungistatic, or fungicidal to the cells of the cell type.

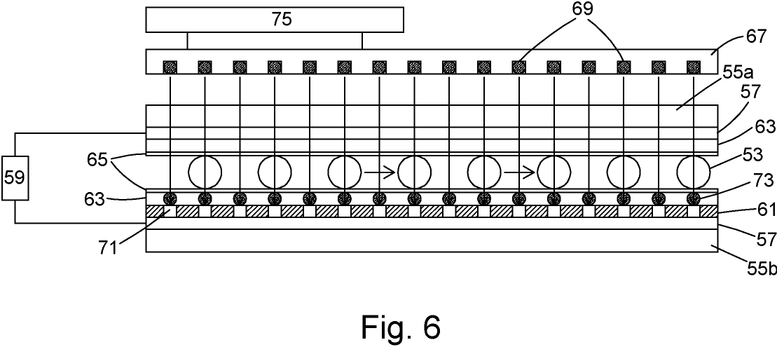

Referring to FIG. 6, an example configuration of a microfluidic chip comprising an oEWOD stack suitable for carrying out methods according to the present invention is illustrated.

The example device is suitable for the manipulation of aqueous microdroplets 53 having been emulsified into an oil, such as a hydrocarbon oil or a fluorocarbon oil, having a viscosity of 5 centistokes or less at 250° C. and which in their unconfined state have a diameter of, for example, between 20 µm and 50 µm. In other embodiments, the device may be suitable for droplets having an unconfined diameter of between 4 µm and 100 µm. In some embodiments, the oil may be the fluorocarbon oil HFE7500 with 2% RAN, or hydrogenated RTM6 containing up to 5% ABIL as well.

The oEWOD stack of the device may comprise top 55a and bottom 55b glass plates, each 500 µm thick and coated with transparent layers of conductive Indium Tin Oxide (ITO) 57 having a thickness of 130 nm. Each of the layers of conductive Indium Tin Oxide (ITO) 57 is connected to an A/C source 59 with the ITO layer on bottom glass plate 55b being the ground. Bottom glass plate 55b is coated with a layer of amorphous silicon 61 which is 800 nm thick. Top glass plate 55a and the layer of amorphous silicon 61 are each coated with a 160 nm thick layer of high purity alumina or Hafnia 63 which are in turn coated with a monolayer of poly(3-(trimethoxysilyl)propyl methacrylate) 65 to render the surfaces of the layer of high purity alumina or Hafnia 63 hydrophobic. In some embodiments, the bottom plate 55b may be made from an opaque material such as silicon.

Top glass plate 55a and the layer of amorphous silicon 61 are spaced 8 µm apart using spacers (not shown) so that the microdroplets undergo a degree of compression when introduced into the device cavity. In some embodiments the distance between 55a and 61 is between 5-50 µm, between 5-40 µm, between 7-30 µm, between 5-20 µm, or between 1-10 µm. The person skilled in the art will understand therefore that the size of the spacers used will depend on the desired size of the distance between 55a and 61. An image of a reflective pixelated screen, illuminated by an LED light source 67 is disposed generally above top glass plate 55a and visible light (wavelength 660 or 830 nm) at a level of 0.01 Wcm2 is emitted from each diode 69 and caused to impinge on the layer of amorphous silicon 61 by propagation in the direction of the multiple downward arrows through top glass plate 55a and the interjacent layers.

At the various points of impingement, photoexcited regions of charge 71 are created in the layer of amorphous silicon 61 which induce modified liquid-solid contact angles on the layer of high purity alumina or Hafnia 63 at corresponding electrowetting locations 73. These modified properties provide the capillary force necessary to propel the microdroplets 53 from one electrowetting location 73 to another. LED light source 67 is controlled by a microprocessor 75 which determines which of the diodes 69 in the array are illuminated at any given time by pre-programmed algorithms.

Advantageously, such a configuration provides a flexible platform for simultaneously manipulating a large number of microdroplets with high accuracy. In particular, said platform is advantageous due to the removal of the need for microdroplets to be manipulated through predetermined pathways, which is a limitation of conventional microfluidic platforms that use arrays of static electrodes or flow pathways to influence the direction in which microdroplets travel. The removal of such limitations enables the complex and precise operations which are necessary for the on-chip sorting of microdroplets and the formation and merging of large arrays for carrying out methods according to the present invention.

Further specific details of microfluidic chips suitable for carrying out the methods of the present invention may be found in our published patent WO 2018/234445, which is herein incorporated by reference.

The device of the present invention may also implement environment control features to create target regions of the chip surface particularly suitable for cell proliferation, such as: controlled temperature, regions of different flow, controlling the carrier fluid to continuously feed cultured cells a supply of nutrients, and control of the local gas concentration in the carrier fluid surrounding the cultured cells. In addition, the carrier fluid can contain a surfactant or a fluorosurfactant. The surfactant can be particularly advantageous because the surfactant can be used to stabilise emulsion droplets. The carrier fluid can be oil. The oil carrier fluid may include fluorocarbon oil such as HFE7500.

For example, the cell culture may be located in a region of low flow and surrounded by regions of faster flow that contain and supply nutrients and chemicals to the culture to encouraging growth. Such mechanisms may form part of the aerobic and anaerobic conditions described in step 11 of FIG. 1 for encouraging proliferation of different cell types by selectively providing conditions favourable for those cell types.

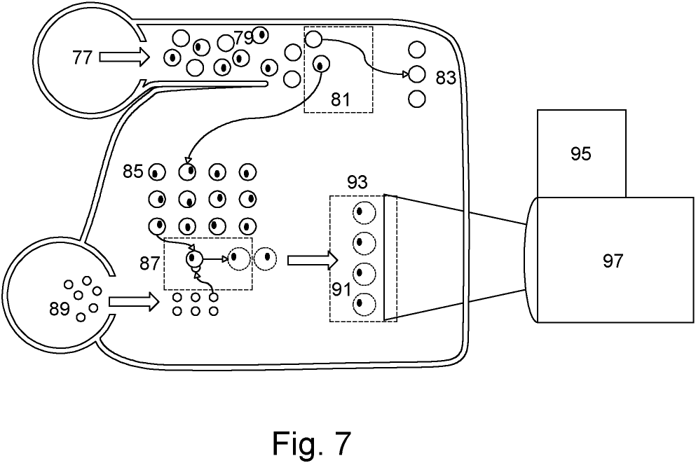

FIG. 7 illustrates an example workflow according to an aspect of the present invention being carried out on the surface of an oEWOD microfluidic chip device.

The view illustrated is of the surface of the oEWOD microfluidic chip, which is configured to simultaneously manipulate a plurality of microdroplets containing respective emulsified cell samples and reagents, via real or virtual electrowetting electrodes, between different locations on the surface.

At the beginning of the workflow, fluid inlet 77 admits an emulsion 79 of a mixture of empty and cell-containing first microdroplets in a fluorocarbon oil carrier fluid.

These first microdroplets are then transferred by means of, for example, oEWOD structures of the chip, to a sorting zone 81 where they are sorted into those which are empty 83 and those which contain cells 85. Thereafter, each of the cell-containing microdroplets 85 is transferred to merging zone 87.

The one or more cells are held in place on the target region for a predefined period of time under conditions which promote cell growth and division within each, forming a colony of cells within the first microdroplets. These first microdroplets then undergo further manipulation, as described previously, to form a first array.

At the end of this period, a second inlet 89 admits second microdroplets. The second microdroplets may be an emulsion of a fluorocarbon oil and one or more medicaments, each microdroplet containing one or more medicaments at a known concentration in an aqueous solution.

The second microdroplets are then merged with the cell-containing first microdroplets 85 at merging zone 87 to form merged microdroplets 91, which are then left for a predefined time. For example, once merged the merged microdroplets may be left to incubate for between 5 and 30 minutes at a temperature of 37° C. During incubation the droplets are monitored via an optical detection system.

The disclosed apparatus thus advantageously allows for the manipulation of microdroplets across a wide range of sizes, and being digitally controlled, provides for dynamically reprogrammable operational steps. The microfluidic substrates of the apparatus have no patterned electrodes, removing several complex low-yield fabrication steps and simplifying the electrical interconnections in comparison to conventional approaches. Device failures caused by dielectric breakdown between neighbouring electrodes are also eliminated thereby.

The resulting device structure permits more elaborate and integrated workflows compared to conventional approaches, such as independent control of the carrier phase and the droplets, as well as allowing for a greater density of droplets to be controlled across regions of the microfluidic chip surface.

### EXPERIMENTAL DETAILS

A number of experimental results obtained using apparatus and methods of the present invention will now be described.

### 1. Bacterial Growth in Droplets

For this experiment the bacteria E.coli DH5-Alpha (with a multicopy vector encoding the green fluorescent protein GFPmut3 and the ampicillin resistance gene (bla) which confers resistance to 100 µg/ml ampicillin) was cultured in a bottle at 37° C. in Luria-Bertani (LB) broth supplemented with 100 µg/ml ampicillin.

To estimate the concentration of bacteria the optical density (OD) of the culture was measured at 600 nm (OD600 = 1.6 to 1.9) and a concentration of 20×10⁶ bacteria per 10 µl was achieved. This corresponds to ^{~}1 cell per 10 µm droplet.

Droplets were produced using vortex emulsification to produce 50 µl of 40 µloil carrier phase/10 µl bacteria suspension.

0.6 µl of an emulsion consisting of Fluorinert™ FC-40 immiscible fluorocarbon oil as the carrier phase (with RAN Fluorosurfactant 5% to encourage droplet formation) plus bacterial suspension was added to the oEWOD device with a 1 µm spacer and incubated.

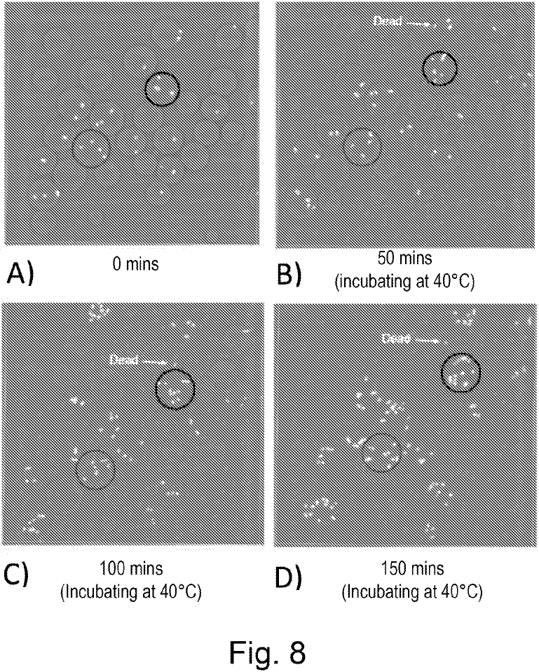

FIG. 8A shows an array comprising a range of droplet sizes with a wide range of starting concentrations (number of bacteria per droplet) at 0 mins.

FIG. 8B shows the same array 50 minutes later following incubation at 40° C. with growth (bacterial replication) visible.

FIG. 8C shows the same array, following incubation for 100 minutes at 40° C. with further growth of bacteria clearly visible when compared with FIG. 8A.

FIG. 8D shows the same array, following incubation for 150 minutes at 40° C. with further growth of bacteria clearly visible.

### 2. Tracking Growth in Droplets

The bacteria growth in the droplets was monitored by optical inspection and three different data analysis methods were used to analyse the optical data:
i. Average intensity - this method involved picking only droplets with one or more bacterial cells and measuring the average fluorescent intensity over the whole droplet.
ii. Fraction of droplet brightness - this method involved picking only droplets with one or more bacterial cells and choosing an intensity threshold above the background but below most of the bacteria. Then the area for which the intensity is greater than the threshold as a fraction of total droplet area is measured.
iii. Counts of spots per droplet - this method involves picking only droplets with one or more bacterial cells, choosing a threshold as above. A spot is defined as a connected area of above-threshold intensity.

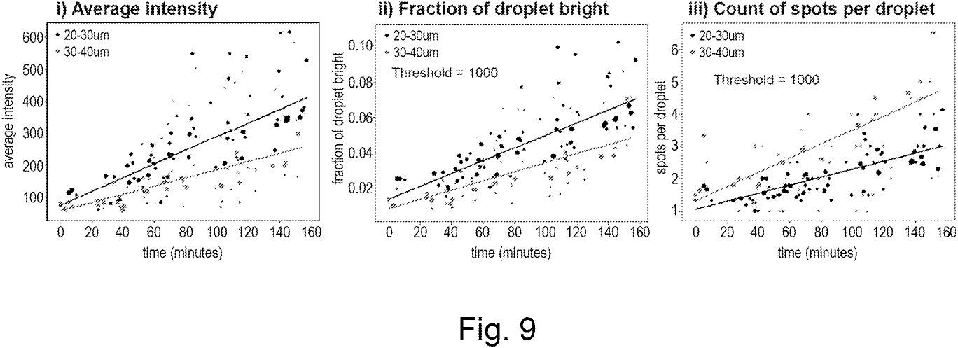

FIG. 9 shows the bacterial growth tracked using each of the three above methods for the previously described experiment. The two colours represent different droplet size ranges; each point plotted is an average over one frame. The spot size is the number of droplets included i.e. small spot denotes few droplets this size in this frame. The rising trend becomes statistically significant (p<0.05) after ^{~}60 min (at this data collect rate; only moderately dependent on analysis method), and reaches p<2e-5 by the end.

### 3. Panel Merge and MIC Determination

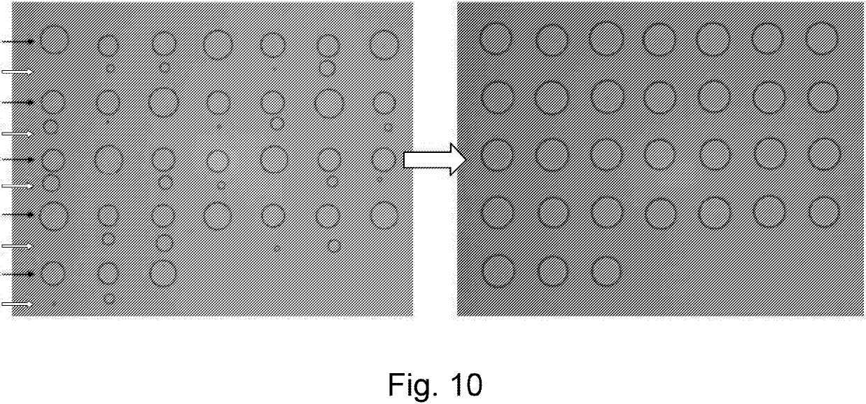

FIG. 10 shows an example of a panel merge performed on a device according to the present invention. Droplets were paired together so that the post-merge droplets were all roughly of uniform size. Every third droplet was labelled as a control and remained unpaired. The smaller droplets in this example contained the antibiotic gentamicin at a concentration of 500 ng/mL in tryptic soy broth (TSB), whilst each of the larger droplets contained 1-3 bacteria in TSB.

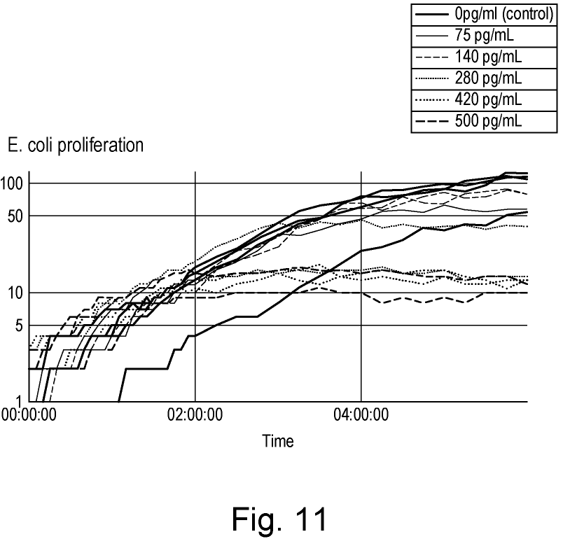

FIG. 11 shows how the concentration of an antibiotic, gentamicin, was reduced to levels where a sample strain of E. coli bacteria showed resistance. Within three hours of reducing the concentration, a robust growth behaviour pattern was observed, allowing for a determination of the MIC. In this particular case, the MIC was determined to lie at approximately 300 pg/mL.

The term “array” as used herein when referring to arrays of microdroplets is intended to refer specifically to microdroplets containing substances to be assayed, and does not include any additional microdroplets which are used as controls but which do not actually undergo testing.

The term medicament, as used herein, means a pharmaceutical formulation, or a candidate pharmaceutical formulation or a candidate pharmaceutical for formulation, containing at least one pharmaceutically active compound.

The term sample/biological sample as used herein means any human, animal, environmental (natural, contrived or modified), or even food sample containing at least one cell type. The sample/biological sample may be selected from: stool, peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper’s fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mammary secretions, mucosal secretion, stool water, pancreatic juice, lavage fluids from sinus cavities, bronchopulmonary aspirates, blastocyl cavity fluid, and umbilical cord blood.

The environmental sample may be selected from: a water sample, an air sample, a soil sample or a surface swab of a surface.

The term bacteriostat/bacteriostatic agent as used herein refers to a medicament that stops bacteria from reproducing, whilst not necessarily killing them. Likewise, the term fungistat/fungistatic agent refers to a medicament that stops fungi from reproducing whilst not necessarily killing them.

The term bactericide/bactericidal agent as used herein refers to a medicament that kills bacteria. Likewise, the term fungicide/fungicidal agent refers to a medicament that kills fungi.

The term aerobic condition(s) as used herein refers to oxygenated conditions within which an aerobic organism can survive and grow.

The term anaerobic condition(s) as used herein refers to conditions of reduced oxygen concentration within which an anaerobic organism can survive and grow.

The term environmental conditions as used herein includes the pH, temperature, pressure, osmolality, salt concentration, type of broth and concentration of any additional nutrient supplements which may be added.

The term classification algorithm as used herein refers to an algorithm capable of automated image analysis. Operations of such algorithms may be as simple as intensity-based threshold determinations, or they may be machine learning operations such as logistic regression, random forest, decision tree, or support vector machines (SVM), or they may be as complex as convolutional neural network (CNN) identification algorithms. Such algorithms are known in the art.

Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

It will further be appreciated by those skilled in the art that although the invention has been described by way of example with reference to several embodiments, it is not limited to the disclosed embodiments and that alternative embodiments could be constructed without departing from the scope of the invention as defined in the appended claims.

1. A method for determining an interaction between a medicament and a cell type, the method comprising:
providing an array of first microdroplets, each containing a cell type derived from a biological sample;
providing an array of second microdroplets, each containing one or more medicaments at one or more predetermined concentrations,
merging the array of first microdroplets and the array of second microdroplets to form an array of merged microdroplets;
monitoring the characteristics of one or more cells in the merged microdroplets using an optical detection system to detect an interaction between a cell type and a medicament.

2. The method of claim 1, wherein the array of second microdroplets comprises microdroplets having a plurality of different concentrations for each medicament.

3. The method of claim 1, wherein the step of providing the array of first microdroplets comprises:
emulsifying the biological sample with an immiscible carrier fluid to form aqueous first microdroplets, at least some of which contain one or more cells of the cell type;
loading the first microdroplets onto a microfluidic chip configured to manipulate the microdroplets using real or virtual electrowetting electrodes;
determining that the cell-containing microdroplets contain at least one viable cell for proliferation; and
creating the array of first microdroplets from the at least one viable cell.

4. The method of claim 1, wherein providing the array of first microdroplets comprises:
moving a first portion of the first microdroplets to a cell proliferation location configured to provide aerobic conditions;
moving a second portion of the first microdroplets to a second cell proliferation location, separate from the first cell proliferation location and configured to provide anaerobic conditions; and
monitoring and recording cell behaviour for the first and second portions of first microdroplets to determine whether the first microdroplets contain viable aerobic cells or viable anaerobic cells.

5. The method of claim 4, wherein the environmental conditions at the first and second cell proliferation locations are selected to promote proliferation of cells of the cell type, the environmental conditions comprising temperature and ambient fluid composition.

6. The method of claim 1, wherein the step of providing the array of second aqueous microdroplets comprises one or more of:
emulsifying one or more medicaments with an immiscible carrier fluid to form aqueous second microdroplets, each microdroplet containing at least one medicament type;
loading the second microdroplets onto a microfluidic chip configured to manipulate the microdroplets using real or virtual electrowetting electrodes;
performing a series of dilution operations on the second microdroplets to create an array.

7. The method of claim 1, wherein the method further comprises performing an assay on a sub-sample of one or more cells from one or more microdroplets.

8. The method of claim 7, wherein the assay is one or more of: a Gram stain, a destructive lysis assay, an oxidase test or a wheat germ agglutinin (WGA) assay.

9. The method of claim 8, wherein the assay is performed prior to merging the first and second arrays.

10. The method of claim 1, wherein the method further comprises performing at least one cell recognition assay on the cells from the biological sample utilising a classification algorithm.

11. The method of claim 10, wherein the cell recognition step comprises analyzing one or more features of the cells from the biological sample, the one or more features comprising at least one of: cell motility, cell shape, and cell proliferation behaviour.

12. The method of claim 1, further comprising: based on characteristics of one or more cells detected in the merged microdroplets overtime, determining a minimum inhibitory concentration (MIC) of at least one medicament for cells of the cell type.

13. The method of claim 12, wherein the medicament is an antibiotic or an antifungal medicament for treating sepsis.

14. The method of claim 13, wherein the method further comprises determining an antibiotic or antifungal treatment regime for treating sepsis based at least in part on the determined MIC.

15. The method according to claim 1, wherein the first and/or second microdroplets further comprises a growth media.

16. The method according to claim 1, further comprising the step of splitting the merged microdroplet containing one or more cells to form a clonal colony.

17. A microfluidic chip device configured to carry out the method of claim 1, the device comprising:
a sorting component configured to separate cell-containing microdroplets from empty microdroplets;
a microdroplet manipulation component configured to manipulate microdroplets using real or virtual electrowetting electrodes;
an optical detection system configured to monitor microdroplets contained in the microfluidic chip via one or more detection windows.

18. The device of claim 17, further comprising a cell-culturing component configured to hold the first microdroplets and provide conditions favourable for cell proliferation.

19. The device of claim 17, further comprising a sample preparation component configured to create a microdroplet emulsion in an immiscible carrier fluid from the biological sample.

20. The device of claim 17, wherein the microdroplet manipulation component includes one or more OEWOD structures comprised of:
a first composite wall comprised of:
a first substrate
a first transparent conductor layer on the substrate, the first transparent conductor layer having a thickness in the range 70 to 250 nm; and
a first dielectric layer on the conductor layer, the first dielectric layer having a thickness in the range 30 to 280 nm;
a second composite wall comprised of:
a second substrate;
a second conductor layer on the substrate, the second conductor layer having a thickness in the range 70 to 250 nm;
a photoactive layer activated by electromagnetic radiation in the wavelength range 400-850 nm on the second conductor layer, the photoactive layer having a thickness in the range 300-1500 nm and
optionally a second dielectric layer on the photoactive layer, the second dielectric layer having a thickness in the range 30 to 280 nm
wherein the exposed surfaces of the first dielectric layer and the second dielectric layer or the photoactive layer are disposed 1-180 µm apart to define a microfluidic space adapted to contain microdroplets;
an A/C source to provide a voltage across the first and second composite walls connecting the first and second conductor layers;
at least one source of electromagnetic radiation having an energy higher than the bandgap of the photoactive layer adapted to impinge on the photoactive layer to induce corresponding virtual electrowetting locations on the surface of the first dielectric layer; and
means for manipulating the points of impingement of the electromagnetic radiation on the photoactive layer so as to vary the disposition of the virtual electrowetting locations thereby creating at least one electrowetting pathway along which the microdroplets may be caused to move.

21. An array of microdroplets, the array comprising:
a plurality of medicament microdroplets, each containing one or more medicaments from a panel of different medicament types at a range of predetermined concentrations; and
a number of control microdroplets which is less than or equal to the number of microdroplets in the plurality of medicament microdroplets, the control microdroplets containing no medicaments.
